(19) United States
(12) Patent Application Publication
(10) Pub. No.: US 2014/0350125 A1
(43) Pub. Date: Nov. 27, 2014

(54) NOVEL OIL-IN-WATER EMULSIONS ENRICHED WITH SALT, WHICH ARE STABILIZED USING NATURAL GUMS, HIGHLY VISCOUS, AND STABLE OVER TIME

(71) Applicant: Emmanuelle Merat, Lautrec (FR)

(72) Inventor: Emmanuelle Merat, Lautrec (FR)

(73) Assignee: SOCIETE D'EXPLOITATION DE PRODUITS POUR LES INDUSTRIES CHIMIQUES SEPPIC, Paris (FR)

(21) Appl. No.: 14/361,102

(22) PCT Filed: Dec. 5, 2012
(86) PCT No.: PCT/FR2012/052803

§ 371 Date: May 28, 2014

(30) Foreign Application Priority Data
Dec. 8, 2011 (FR) 1161304

(51) Int. Cl. A61K 8/81; A61K 8/92; A61K 8/20; A61Q 5/02; A61Q 19/00; A61Q 17/04; A61Q 19/10; A61K 8/73; A61Q 5/00

(52) U.S. Cl. CPC A61K 8/8188; A61K 8/73; A61K 8/92; A61K 8/20; A61Q 5/002; A61Q 19/00; A61Q 17/04; A61Q 19/10; A61Q 5/02
USPC 514/772.4; 510/159; 510/127

(57) ABSTRACT

An oil-in-water emulsion includes: 5 to 55 wt % of an oil phase consisting of at least one oil and/or one wax; 0.06 to 4.5 wt % of at least one cross-linked anionic polyelectrolyte resulting from the polymerization of at least one monomer having a strong acid function, the monomer being partially or totally salified 2-methyl 2-[(1-oxo 2-propenyl)amino] 1-propanesulfonic acid, with at least one neutral monomer selected from the N,N-dialkyl acrylamides, wherein each of the alkyl groups include between one and four carbon atoms, and at least one monomer of formula (I), where R is a straight or branched alkyl radical including eight to twenty carbon atoms and 1≦n≦20, in the presence of at least one cross-linking agent; and 0.0025 to 1 wt % of xanthan gum; 0.0025 to 1 wt % acacia gum; 38.5 to 94.835 wt % of a cosmetically acceptable aqueous phase.

The invention relates to novel oil-in-water emulsions, as well as use thereof in cosmetics and pharmaceutics.

Cosmetic compositions presented in the form of oil-in-water emulsions marketed by the cosmetics industry and by the pharmaceutical industry very frequently comprise synthetic thickening polymers for increasing the viscosity of said oil-in-water emulsions which may be presented in the form of creams, lotions and which are applied directly to the skin.

These synthetic thickening polymers make it possible to thicken the aqueous phases present in said oil-in-water emulsions, thus obtaining either the desired consistency or a stabilisation effect of said emulsion.

The synthetic thickening polymers currently used in these fields are presented in two physical forms, powder form and liquid form for which the polymer is prepared by inverse emulsion radical polymerisation using surfactants, and commonly referred to as inverse latex.

Among the best known synthetic thickening polymers presented in powder form, powder form, mention can be made of polymers based on acrylic acid or copolymers based on acrylic acid and the esters thereof. Mention may be made for example of the polymers marketed under the brand name CARBOPOL™ and PEMULEN™. They are described in particular in the American patents U.S. Pat. No. 5,373,044 and U.S. Pat. No. 2,798,053 and in European patent EP 0 301 532.

In cosmetics, homopolymers or copolymers based on 2-acrylamido-2-methyl-propane sulfonic acid and/or salts thereof are also used, again in powder form. These thickening polymers are marketed under the brand name Aristoflex™ and described in particular in the European patents EP 816 403, EP 1 116 733 and EP 1 069 142. These synthetic thickeners in powder form are obtained by precipitation polymerisation; the monomer(s) is (or are) placed in solution in an organic solvent such as benzene, ethyl acetate, cyclohexane, tertio-butanol; this method therefore requires numerous successive steps for purifying the end product, to remove any trace of residual solvent.

The cosmetics and pharmaceutical industries also very widely use thickeners presented in the form of inverse latexes and in particular those marketed by the applicant. Mention may be made for example of the thickeners Sepigel™ 305, Simulgel™ 600, Simulgel™ EG, Simulgel™ EPG, Simulgel™ NS, Simulgel™ A, Sepiplus™ 400, Sepiplus™ 250 and Sepiplus™ 265. These thickeners are obtained by inverse emulsion radical polymerisation. They have the advantage of being easier to handle, in particular at ambient temperature, and disperse very quickly in water. Furthermore, these products develop remarkably high thickening performances; these performances are probably the consequence of the method used for the preparation thereof, a dispersed phase radical polymerisation reaction, which results in polymers with very high molecular weights.

Nevertheless, these synthetic thickeners presented in the form of inverse latex contain an oil, and one or a plurality of surfactants which may sometimes induce skin intolerance reactions on particularly sensitive subjects; in addition this presence of oil makes them unusable for the preparation of clear aqueous gels.

The applicant has therefore developed synthetic thickeners having thickening performances equivalent or superior to inverse latexes, but better tolerated by the skin, in particular due to the absence of any oil phase that may lead to clearer aqueous gels. These products are presented in the form of powder but have dissolution times, and therefore ease of use, comparable to those of products in the form of liquids. These compounds, described in the European patent application published under the number EP 1 496 081, are obtained by the conventional polymerisation techniques, such as dispersed phase radical polymerisation, inverse suspension radical polymerisation, inverse emulsion or inverse microemulsion radical polymerisation. The synthetic thickening systems obtained are then extracted and purified by various techniques such as precipitation in a separate solvent, precipitation in a separate solvent optionally followed by washing, drying by atomisation or by azeotropic dehydration, optionally followed by washing by a carefully chosen solvent. These synthetic thickeners therefore combine some of the advantages of the synthetic thickeners in the form of conventional powders (absence of oil, obtaining of clearer aqueous gels) and the advantages of synthetic thickeners presented in the form of inverse latexes (high dissolution rate, remarkable thickening capacity and stabilising properties). However, for some uses, customers using such synthetic thickening systems wish to be able to manufacture gels that are even clearer than those obtained at the present time, or even transparent gels. In addition, the gels obtained with these synthetic thickeners do not have satisfactory stability when the composition is enriched with electrolytes, as is often the case with compositions comprising sun filters and/or coloured pigments and/or plant extracts enriched with electrolytes.

The applicant has also developed synthetic thickening systems such as those described in the French patent application published under the number 2 910 899, which discloses a linear, branched or cross-linked terpolymer of at least one monomer having a free, partially salified or completely salified strong acid function, with at least one neutral monomer, and at least one monomer of formula (A):

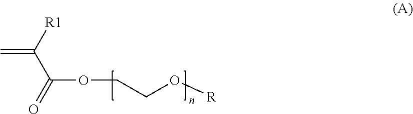

Wherein R1 represents a hydrogen atom or a methyl radical, R represents a linear or branched alkyl radical comprising from eight to thirty carbon atoms and n represents a number greater than or equal to one and less than or equal to fifty. These polymers have very marked thickening properties, in particular in the presence of electrolytes. They function over a wide pH range and make it possible to produce transparent gels. However, formulations with a low pH thickened by some of them do not have satisfactory resistance to salts over the long term and some of them, which contain fatty alcohols, have an unappealing elastic appearance and give sticky sensations to the touch and/or an appearance of a granular and non-continuous cream or emulsion.

The applicant has shown that these drawbacks could be avoided by selecting some of these terpolymers, which had not been disclosed in the French patent application published under the number 2 910 899, and has developed novel branched or cross-linked anionic polyelectrolytes, such as those described in the international application published under the number WO 2011/030044, which result from the radical polymerisation of at least one monomer having a partially salified or completely salified strong acid function, with at least one neutral monomer, and at least one monomer of formula (B):

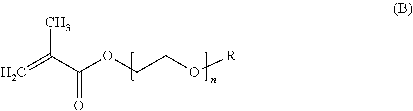

wherein R represents a linear or branched alkyl radical comprising eight to twenty carbon atoms and n represents a number greater than or equal to one and less than or equal to thirty.

The cosmetics and pharmaceutical industries are also seeking galenic forms reducing the risks of skin intolerances, and consequently tend to select ingredients constituting said galenic forms that are well tolerated and also to reduce, in the composition thereof, the proportion of ingredients liable to increase the likelihood of intolerance reactions on the skin. In this regard, the cosmetics and pharmaceutical industries seek to develop oil-in-water emulsions that are devoid of stabilising systems comprising emulsifying surfactants. The terpolymers described in the international application published under the number WO 2011/030044 therefore constitute ideal candidates for preparing oil-in-water emulsions free from emulsifying surfactants.

However, when oil-in-water emulsions are prepared using such synthetic thickening terpolymers in the absence of emulsifying surfactants, the appearance of lumps is observed in said oil-in-water emulsions free from emulsifying surfactants during the storage thereof over time. It is therefore necessary to develop novel oil-in-water emulsions, free from emulsifying surfactants, that do not present during the long-term storage thereof clusters on storage, but which retain a high viscosity in the presence of electrolyte-enriched media and over a wide pH range, as well as satisfactory sensory properties, namely free from a sticky and stringy character on the handling thereof and after application on the skin.

Polysaccharides have been used for many years as agents for modifying texture and/or rheology for preparing food, cosmetic or pharmaceutical compositions. Depending on the chemical constitution thereof, they may be used as gelling agents and/or as thickening agents. Thickening agent means a chemical compound that increases the viscosity of the medium wherein it is introduced. Gelling agent means a compound that transforms a liquid medium into a structured state, which does not flow, by forming a three-dimensional lattice in the liquid; the gel being considered to be an intermediate state between the liquid state and the solid state.

Polysaccharides are polymers of saccharides. The IUPAC definition of saccharides designates sugars, compounds of sugars strictly speaking and derivatives thereof obtained either by reduction of a carbonyl group, or by oxidation of one or more hydroxyl functions, or by replacing one or more hydroxyl functions with a hydrogen atom, an amine group, a phosphate function or a sulfate function.

The polysaccharides most commonly used for preparing food, cosmetic or pharmaceutical compositions mainly consist of sugars, such as glucose, galactose or mannose or sugar derivatives for which the hydroxyl function of the terminal carbon has been oxidised into a carboxyl function. Two distinct groups can be distinguished among polysaccharides: polysaccharides consisting solely of (or poly-sugars) and polysaccharides consisting of sugar derivatives.

Among the polysaccharides consisting of sugar derivatives, one may distinguish:
sulfated galactans, which are polymers of galactose that may have appended ester-sulfate groups, represented in particular by algal polyosides such as carrageenans and agar;
uronans, which are polymers of uronic acids such as algins and pectins;
heteropolymers of sugars and uronic acids: these polymers are normally found in exudates of sap (such as for example exudate of gum arabic and exudate of karaya gum), but they are also produced by microorganisms, such as for example xanthan gum and gellan gum;
glucosaminoglycans, which are polyosides formed from a glucose derived by replacing its hydroxyl on C-2 with an amine (called 2-amino-2-desoxy-D-glucose or glucosamine). The amine function may moreover be acetylated. Among the hydrocolloids in this class there are chitosan formed solely from glucose amine units, and hyaluronan, the repetition unit of which is a dimer of glucosamine and glucuronic acid.

Xanthan gum (GX) has for the past few decades become the microbial polyoside most used in industry. Xanthan is a polysaccharide synthesised by bacteria of the genus Xanthomonas and, commercially, only the species X. campestris is used. The main chain of (GX) is identical to that of cellulose, that is to say it is formed by β-D-glucose units connected by carbon atoms 1 and 4. There is a branched triholoside every two glucose units in the main chain, in a regular alternating fashion; each branching consisting of a triholoside composed of two mannoses and a glucuronic acid, of the type: β-D-Manp-(1→4)-β-D-GlcAp-(1→2)-α-D-Manp-(1→3) [I. Capron et al., “About the native and renaturated conformation of xanthan exopolysaccharide”. 1997).

Xanthan gum (GX) is available in the form of a sodium, potassium or calcium salt.

Acacia gum is a branched complex polysaccharide the main chain of which consists of units of β-D-galactose interconnected by carbon atoms 1 and 3. The chains branched to the main chain consist of units of β-D-galactose interconnected by the carbon atoms 1 and 6, also bearing α-arabinose units, and in small proportions β-glucoronosyl units. Both the main chain and the pendant chains contain α-L-arabinosyl, α-L-rhamnopyranosyl, β-D-glucuronopyranosyl and 4-O-methyl-β-D-glucuronopyranosyl units. As a subcategory of polysaccharides, and more particularly as heteropolymers of sugars and uronic acids, xanthan gum and acacia gum have already been associated with synthetic thickeners resulting from the radical polymerisation of monomers such as acrylic acid, acrylic acid esters, 2-acrylamido-2-methyl-propanesulfonic acid and/or salts thereof, acrylamide and 2-hydroxyethyl acrylate.

The French patent application published under the number 2 940 111 describes the use of compositions comprising polysaccharides, which may be associated with hydrophilic gelling agents in particular chosen from among copolymers comprising 2-acrylamido-2-methylpropane sulfonic acid and acrylamide as constituent monomers, or copolymers comprising 2-acrylamido-2-methylpropane sulfonic acid and polyoxethylenated alkyl methacrylates. These compositions are intended for uses in make-up, having the property of not transferring onto the substrates whereon they are placed in contact as well as the property consisting of resisting water after application on the skin. However, the hydrophilic gelling agents described in the French patent application published under the number 2 940 111 are known not to make it possible to reach high levels of viscosity in the presence of media enriched with electrolytes.

The inventors have therefore sought to develop novel oil-in-water emulsions free from emulsifying surfactants in the stabilising system thereof, enriched with salts, retaining a high viscosity and a homogeneous appearance after a prolonged storage period.

For this reason, according to a first aspect, the subject matter of the invention is a composition (C₁) presented in the form of an emulsion of the oil-in-water type, characterised in that it comprises, for 100% of the weight thereof:
from 5% to 55% by weight, more particularly from 7% to 30% by weight and even more particularly from 10% to 20% by weight of an oil phase (P₁) consisting of at least one oil and optionally at least one wax;
from 0.06% to 4.5% by weight, more particularly from 0.3% to 3.6% by weight, and even more particularly from 0.3% to 2.7% by weight of at least one cross-linked anionic polyelectrolyte (PA) resulting from the polymerisation of partially or completely salified 2-methyl 2-[(1-oxo 2-propenyl)amino] 1-propane sulfonic acid, with at least one neutral monomer chosen from the N,N-dialkyl acrylamides, wherein each of the alkyl groups comprises between one and four carbon atoms, and at least one monomer of formula (I):

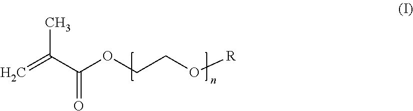

wherein R represents a linear or branched alkyl radical comprising eight to twenty carbon atoms and n represents a number greater than or equal to one and less than or equal to twenty, in the presence of at least one cross-linking agent;
from 0.0025% to 1% by weight, more particularly from 0.0125% to 0.8% by weight, and even particularly from 0.0125% to 0.6% by weight of a xanthan gum gum (GX);
from 0.0025% to 1% by weight, more particularly from 0.0125% to 0.8% by weight, and even more particularly from 0.0125% to 0.6% by weight of an acacia gum gum (GA);
from 38.5% to 94.935% by weight, more particularly from 55% to 94.935% by weight, and even more particularly from 65% to 94.935% by weight of a cosmetically acceptable aqueous phase (P₂), said aqueous phase (P₂) comprising, for 100% of the weight thereof, from 1% to 10% by weight, more particularly from 1% to 8% by weight, and even more particularly from 1% to 4% by weight of at least one salt (S) presented in a dissolved form;

said composition (C₁) furthermore being characterised in that the ratio by weight between the xanthan gum (GX) and the acacia gum (GA) is greater than or equal to 1/3 and less than or equal to 3/1, more particularly greater than or equal to 1/3 and less than or equal to 3/2, and even more particularly greater than or equal to 1/3 and less than or equal to 1/1.

“Oils” means in the present application the compounds and/or mixtures of compounds insoluble in water, being in a liquid aspect at a temperature of 25° C. Among the oils that can be used in the oil phase (P₁) of the composition (C₁) that is the subject matter of this invention, the following can be mentioned:
mineral oils such as paraffin oil, liquid petrolatum, isoparaffins or white mineral oils;
oils of animal origin, such as squalene or squalane;
plant oils, such as phytosqualane, sweet almond oil, copra oil, castor oil, jojoba oil, olive oil, rapeseed oil, peanut oil, sunflower oil, wheat germ oil, maize germ oil, soya bean oil, cotton oil, alfalfa oil, poppy oil, pumpkin seed oil, evening primrose oil, millet oil, barley oil, rye oil, safflower oil, candlenut oil, passionflower oil, hazelnut oil, palm oil, shea butter, apricot kernel oil, calophyllum oil, sisymbrium oil, avocado oil, calendula oil, oils derived from flowers or vegetables;
ethoxylated plant oils;
synthetic oils such as fatty acid esters such as butyl myristate, propyl myristate, cetyl myristate, isopropyl palmitate, butyl stearate, hexadecyl stearate, isopropyl stearate, octyl stearate, isocetyl stearate, dodecyl oleate, hexyl laurate, propyleneglycol dicaprylate, esters derived from lanolic acid, such as isopropyl lanolate, isocetyl lanolate, monoglycerides, diglycerides and triglycerides of fatty acids such as glycerol triheptanoate, alkybenzoates, hydrogenated oils, poly(alpha-olefins), polyolefins such as polyisobutene, synthetic isoalkanes, such as isohexadecane, isododecane, perfluorinated oils and
silicone oils such as dimethylpolysiloxanes, methylphenyl-polysiloxanes, silicones modified by amines, silicones modified by fatty acids, silicones modified by alcohols, silicones modified by alcohols and fatty acids, silicones modified by polyether groups, modified epoxy silicones, silicones modified by fluorinated groups, cyclic silicones and silicones modified by alkyl groups.

“Waxes” means in the present application compounds and/or mixtures of compounds that are insoluble in water, with a solid appearance at a temperature greater than or equal to 45° C. Among the waxes that can be used in the oil phase (P₁) of the composition (C₁) that is the subject matter of this invention, the following can be cited: beeswax, carnauba wax, candelilla wax, ouiricury wax, Japan wax, cork fibre wax, sugarcane wax, paraffin waxes, lignite waxes, microcrystalline waxes, lanolin wax; ozokerite; polyethylene wax, silicone waxes; plant waxes; fatty alcohols and fatty acids solid at ambient temperature; glycerides solid at ambient temperature.

Among the other fats that can be associated with the oil phase (P₁) of the composition (C₁) that is the subject matter of this invention, saturated or unsaturated fatty alcohols, linear or branched, or saturated or unsaturated fatty acids, linear or branched, can be be cited.

In the composition (C₁) that is the subject matter of this invention, xanthan gum (GX) means a heteropolymer of oses and uronic acids, obtained by the aerobic fermentation of bacteria of the genus Xanthomonas campestris. Its structure consists of a main chain of β-D-glucose units interconnected by the carbon atoms 1 and 4. A branched triholoside is counted every two glucose units in the main chain, in a regular alternating fashion; each branch consisting of a triholoside composed of two mannoses and a glucuronic acid, of the type: β-D-Manp-(1→4)-β-D-GlcAp-(1→2)-α-D-Manp-(1→3).

Xanthan gums (GX) are available in the form of a sodium, potassium or calcium salt, and are characterised by a molecular weight of between 1,000,000 to 50,000,000. Xanthan gums are represented for example by the product sold under the trade name Rhodicare™ by the company Rhodia Chimie and under the trade name Keltrol™ CG-T by the company CP-KELCO.

In the composition (C₁) that is the subject matter of this invention, acacia gum (GA) means a heteropolymer of sugars and uronic acids, a branched complex the main chain of which consists of units of β-D-galactose interconnected by the carbon atoms 1 and 3. The chains branched to the main chain consist of units of β-D-galactose interconnected by the carbon atoms 1 and 6, also carrying units of α-arabinose, and in smaller proportions β-glucoronosyl units. Both the main chain and the hanging chains contain α-L-arabinosyl, α-L-rhamnopyranosyl, β-D-glucuronopyranosyl and 4-O-methyl-β-D-glucuronopyranosyl units.

Acacia gum (GA) is also designated by the term “gum arabic” and constitutes a solidified descending sap exudate, amalgamated naturally or by incision of the trunk or at the foot of trees in the acacia family.

The acacia gum (GA) used in this invention is represented for example by the product sold under the trade name Efficacia™ M by the company Colloïdes Naturels International.

Cross-linked anionic polyelectrolyte (PA) means, in the definition of the composition (C₁) that is the subject matter of this invention, a non-linear cross-linked anionic polyelectrolyte, presented in the state of a three-dimensional lattice insoluble in water, but swellable in water, but swellable in water and leading to the obtaining of a chemical gel.

Partially salified or completely salified means in the definition of the cross-linked anionic polyelectrolyte (PA) present in the composition (C₁) as defined above, that said 2-methyl 2-[(1-oxo 2-propenyl)amino] 1-propane sulfonic acid is partially or completely salified, generally in the form of alkaline metal salt, such as for example sodium salt or potassium salt, or in the form of ammonium salt.

Said cross-linked anionic polyelectrolyte (PA) used in the composition C₁, as defined above comprises generally between 5% molar and 95% of the monomer from 2-methyl 2-[(1-oxo 2-propenyl)amino] 1-propane sulfonic acid, more particularly between 10% molar and 90% molar, more particularly between 20% molar and 80% molar, and even more particularly between 60% molar and 80% molar.

Said cross-linked anionic polyelectrolyte (PA) used in the composition C₁ as defined above comprises generally between 4.9% molar and 90% molar of neutral monomer chosen from the N,N-dialkyl acrylamides, wherein each of the alkyl groups comprise between one and four carbon atoms, more particularly between 9.5% molar and 85% molar, more particularly between 15% and 75% molar, and even more particularly between 15% molar and 39.5%

Said cross-linked anionic polyelectrolyte (PA) used in the composition (C₁) as defined above comprises generally between 0.1% molar and 10% molar of monomers of formula (I) and more particularly between 0.5% molar and 5% molar.

In the definition of said cross-linked anionic polyelectrolyte (PA) used in the composition C₁ as defined above the neutral monomer is more particularly chosen from the N,N-dialkyl acrylamides, wherein each of the alkyl groups comprises between one and four carbon atoms and is in particular chosen from N,N-dimethyl acrylamide, N,N-diethyl acrylamide and N,N-dipropyl acrylamide.

In the definition of said cross-linked anionic polyelectrolyte (PA) used in the composition C₁ such as defined above, linear or branched alkyl radical comprising from eight to twenty carbon atoms means more particularly in formula (I) for R:
either a radical derived from linear primary alcohols such as for example, the octyl, decyl, undecyl, dodecyl, tridecyl, tetradecyl, pentadecyl, hexadecyl, heptadecyl, octadecyl, nonadecyl or eicosyl radical:
or a radical derived from Guerbet alcohols, which are branched 1-alkanols complying with the general formula:

CH₃—(CH₂)ₚ—CH[CH₃—(CH₂)ₚ₋₂]—CH₂OH,

wherein p represents an integer number between 2 and 9, such as, for example, the 2-ethyl hexyl, 2-propyl heptyl, 2-butyl octyl, 2-pentyl nonyl, 2-hexyl decyl or 2-octyl dodecyl radicals;
or a radical derived from the isoalkanol complying with the general formula;

CH₃—CH(CH₃)—(CH₂)ₘ—CH₂OH,

wherein m represents an integer number between 2 and 16, such as, for example, the 4-methyl pentyl, 5-methyl hexyl, 6-methyl heptyl, 15-methyl pendadecyl or 16-methyl heptadecyl radicals, or the 2-hexyl octyl, 2-octyl decyl or 2-hexyl dodecyl radicals.

According a particular aspect, the subject matter of the invention is a composition (C₁) as defined above, characterised in that said cross-linked anionic polyelectrolyte (PA) comprises, for 100% molar of its constituent monomers:
from 20% molar to 80% molar of monomeric units issuing from the monomer comprising a partially or completely salified strong acid function;
from 15% molar to 75% molar of monomeric units issuing from a neutral monomer chosen from the N,N-dialkyl acrylamides, wherein each of the alkyl groups comprises between one and four carbon atoms;
from 0.5% to 5% molar of monomeric units issuing from a monomer of formula (I) as defined above.

According to another particular aspect, the subject matter of the invention is a composition (C₁) such as defined above, characterised in that, in the definition of said crosslinked anionic polyelectrolyte (PA), said neutral monomer is N,N-dimethyl acrylamide.

According to a particular aspect of this invention, the subject matter of the invention is a composition (C₁) such as defined previously, characterised in that, in the definition of said crosslinked anionic polyelectrolyte (PA), in formula (I) R designates more particularly an alkyl radical comprising 12 to 18 carbon atoms.

According to another particular aspect, the subject matter of the invention is a composition (C₁) such as defined above, characterised in that, in the definition of said cross-linked anionic polyelectrolyte (PA), in formula (I) n designates more particularly an integer number between 3 and 20.

According to an even more particular aspect the subject matter of the invention is a composition (C₁) such as defined above, characterised in that, in the definition of said cross-linked anionic polyelectrolyte (PA), said monomer of formula (I) is tetraethoxylated lauryl methacrylate.

According to an even more particular aspect the subject matter of the invention is a composition (C₁) such as defined above, characterised in that, in the definition of said cross-linked anionic polyelectrolyte (PA), said monomer of formula (I) is eicosaethoxylated stearyl methacrylate.

According to another particular aspect, the subject matter of the invention is a composition (C₁) such as defined above, wherein said cross-linked anionic polyelectrolyte (PA) is cross-linked with a diethylenic or polyethylenic compound in the molar proportion expressed in relation to the monomers used, from 0.005% to 1%, more particularly from 0.01% to 0.5% and quite particularly from 0.01% to 0.25%. The cross-linking agent is more particularly chosen from ethylene eglycol dimethacrylate, tetraallyloxyethane, ethylene glycol diacrylate, diallyl urea, triallyl amine, trimethylol propanetriacrylate or methylene-bis(acrylamide) or a mixture of these compounds.

The cross-linked anionic polyelectrolyte (PA) used in the composition (C₁) as defined previously may also comprise various additives, such as complexing agents, transfer agents or chain-limiting agents.

According to a particular aspect, the subject matter of the invention is a composition (C₁) as described above wherein said cross-linked anionic polyelectrolyte (PA) is chosen from the terpolymers of 2-methyl 2-[(1-oxo 2-propenyl)amino] 1-propane sulfonic acid partially salified in the form of ammonium, N,N-dimethyl acrylamide and tetraethoxylated lauryl methacrylate cross-linked with trimethylol propanetriacrylate, or the terpolymers of 2-methyl 2-[(1-oxo 2-propenyl)amino] 1-propanesulfonic acid partially salified in the form of ammonium salt, N,N-dimethyl acrylamide and eicosaethoxylated stearyl methacrylate, cross-linked with trimethylol propanetriacrylate.

According to an even more particular aspect, the subject matter of the invention is a composition (C₁) as described above wherein said crosslinked anionic polyelectrolyte (PA) is a terpolymer of 2-methyl 2-[(1-oxo 2-propenyl)amino] 1-propanesulfonic acid partially salified in the form of ammonium, N,N-dimethyl acrylamide and tetraethoxylated lauryl methacrylate, cross-linked with trimethylol propanetriacrylate.

According to an even more particular aspect, the subject matter of the invention is a composition (C₁) as described above wherein said crosslinked anionic polyelectrolyte (PA) comprises, for 100% molar
from 60% molar to 80% molar of monomeric units issuing from 2-methyl 2-[(1-oxo 2-propenyl)amino] 1-propanesulfonic acid partially salified in the form of ammonium,
from 15% molar to 39.5% molar of monomeric units issuing from N,N-dimethyl acrylamide, and
from 0.5% molar to 5% molar of monomeric units issuing from tetraethoxylated lauryl methacrylate.

In composition (C₁) according to the invention, the in situ combination of the cross-linked anionic polyelectrolyte (PA), xanthan gum (GX) and acacia gum (GA), as defined above and in the proportions as defined previously, constitutes the stabilising system of said composition (C₁).

The expression “cosmetically acceptable” used in the definition of the aqueous phase (P₂) of the composition (C₁) that is the subject matter of this invention, means according to European Economic Community Council Directive No. 76/768/EEC of 27 Jul. 1976 amended by Directive No 93/35/EEC of 14 Jun. 1993, that said aqueous phase (P₂) comprises water and any substance or preparation intended to be placed in contact with the various parts of the human body (epidermis, pilous and hair system, nails, lips and genital organs) or with the teeth and the mucosa of the oral cavity with a view exclusively and mainly to cleansing them, perfuming them, modifying the appearance thereof and/or correcting the body odours thereof and/or protecting them or keeping them in good condition.

A cosmetically acceptable aqueous phase (P₂) included in the composition (C₁) that is the subject matter of this invention contains water, may conventionally contain one or a plurality of cosmetically acceptable organic solvents, a mixture of water and one or a plurality of cosmetically acceptable organic solvents. The cosmetically acceptable solvents may more particularly be chosen from the polyhydric alcohols such as for example glycerol, diglycerol, triglycerol, glycerol oligomers, xylitol, erythritol, sorbitol, 2-methyl-1,3-propanediol; alkoxylated polyhydric alcohols; glycols, such as for example butylene glycol, hexylene glycol, caprylyl glycol or 1,2 octanediol, pentylene glycol or 1,2 pentanediol, monopropylene glycol, dipropylene glycol, isoprene glycol, butyldiglycol, polyethylene glycols with a molecular weight of between 200 g·mol⁻¹ and 8000 g·mol⁻¹; or water-soluble alcohols such as for example ethanol, isopropanol or butanol.

In the composition (C₁), as defined above, salt (S) means a heteropolar compound the crystalline lattice of which comprises the participation of at least one type of cation different to the hydrogen ions and at least one type of anion different to the hydroxide ions.

According to a particular aspect, the salt (S) presented in dissolved form in the aqueous phase (P₂) of the composition (C₁) that is the subject matter of this invention is selected from the inorganic salts and from the organic salts.

According to this particular aspect, the salt (S) is particularly selected from inorganic salts.

According to a more particular aspect, the subject matter of the invention is a composition (C₁) as defined above, characterised in that the salt (S) is an inorganic salt consisting of a cation that is the ammonium ion or a metal cation and an anion selected from the elements of the group consisting of the halide ions, the carbonate ions, the bicarbonate ions, the phosphate anions, the nitrate anions, the borate anions and the sulfate anions.

According to a more particular aspect, the subject matter of the invention is a compositon (C₁) as defined above, characterised in that the salt (S) is an inorganic salt the metal cation of which is a monovalent or multivalent cation chosen from the elements of the group consisting of the sodium, potassium, lithium, calcium, magnesium, zinc, manganese, iron, copper, cobalt, silver, gold, aluminium, barium, bismuth, selenium, zirconium, strontium and tin cations.

According to an even more particular aspect, the subject matter of the invention is a composition (C₁) as defined above characterised in that the salt (S) is an inorganic salt chosen from the elements of the group consisting of sodium chloride, calcium chloride, magnesium chloride, calcium sulfate, ammonium sulfate, calcium carbonate, zinc sulfate, magnesium sulfate, sodium borate.

According to another particular aspect, the salt (S) is particularly selected from organic salts.

According to a particular aspect, the subject matter of the invention is a composition (C₁) as defined above, characterised in that the salt (S) is an organic salt consisting of a cation that is the ammonium ion or a metal cation and an organic ion that is an organic compound having at least one carboxylic acid function in carboxylate form or at least one sulfonic acid function in sulfonate form or at least one sulfate function.

According to this particular aspect, the subject matter of the invention is a composition (C₁) as defined above characterised in that the salt (S) is an organic salt consisting of a monovalent or multivalent metal cation more particularly chosen from the elements of the group consisting of the sodium, potassium, lithium, calcium, magnesium, zinc, manganese, iron, copper, cobalt, silver, gold, aluminium, barium, bismuth, selenium, zirconium, strontium and tin cations. According to this particular aspect, the salt (S) is an organic salt consisting of the cation chosen from the elements of the group consisting of the sodium, calcium, magnesium, zinc and manganese cations, and even more particularly the salt (S) is an organic salt consisting of the sodium cation.

According to a particular aspect, the subject matter of the invention is a composition (C₁) as defined above characterised in that the salt (S) is an organic salt consisting of a cation that is the ammonium ion or a metal cation as described above, and an organic ion that is an organic compound having at least one carboxylic acid function in carboxylate form chosen from the elements of the group consisting of glycolic acid, citric acid, tartric acid, salicylic acid, lactic acid, mandelic acid, ascorbic acid, pyruvic acid, fumaric acid, retinoic acid, benzoic acid, kojic acid, malic acid, gluconic acid, galacturonic acid, proprionic acid, heptanoic acid, 4-amino benzolic acid, cinnamic acid, benzalmalonic acid, aspartic acid and glutamic acid.

According to an even more particular aspect, the subject matter of the invention is a composition (C₁) as defined above characterised in that the salt (S) is an organic salt selected from the elements in the group consisting of sodium glycolate, sodium citrate, sodium salicylate, sodium lactate, sodium gluconate, zinc gluconate, manganese gluconate, copper gluconate and magnesium aspartate.

According to another particular aspect, the subject matter of the invention is a composition (C₁) as defined above, characterised in that the salt (S) is an organic salt consisting of a cation that is the ammonium ion or a metal cation as described above, and an organic anion that is an organic compound having at least one sulfonic acid function in sulfonate form chosen from the elements of the group consisting of 2-phenylbenzimidazole-5-sulfonic acid, the sulfonic acids derived from benzophenones, such as for example 4-hydroxy-2-methoxy 5-(oxo-phenylmethyl)benzenesulfonic acid (said acid being registered under the name Benzophenone-4), the sulfonic acids derived from 3-benzylidene camphor such as for example 4-(2-oxo-3-bornylidenemethyl)benzene sulfonic acid, 2-methyl 5-(2-oxo-3-bornylidenemethyl)benzene sulfonic acid.

According to an even more particular aspect, the subject matter of the invention is a composition (C₁) as defined above characterised in that the salt (S) is an organic salt selected from the elements of the group consisting of sodium 2-phenylbenzimidazole-5 sulfonate and sodium 4-hydroxy 2-methoxy 5-(oxo-phenylmethyl)benzene sulfonate.

2-phenyl benzimidazole-5-sulfonic acid is marketed in particular under the brand name EUSOLEX™232 by the company Merck. Sodium 4-hydroxy-2-methoxy-5-(oxo-phenylmethyl)benzene sulfonate is registered under the name Benzophenone-5.

In general terms, the composition (C₁) that is the subject matter of this invention comprises, in addition to said oil phase (P₁), the stabilising system composed of the in situ combination of the cross-linked anionic polyelectrolyte (PA), acacia gum (GA) and xanthan gum (GX) as defined above, and said cosmetically acceptable aqueous phase (P₂) as defined above adjuvants and/or additives routinely used in the field of cosmetic, dermocosmetic, pharmaceutical and dermopharmaceutical formulations.

Among the adjuvants likely to be present in the compositions (C₁) that are the subject matter of this invention, the following can be cited: film-forming compounds, hydrotropic agents, plasticizing agents, opacifying agents, pearlescent agents, superfatting agents, sequestering agents, chelating agents, non-ionic detergent surfactants, antioxidant agent, perfumes, preservatives, conditioning agents, bleaching agents intended for decolouring hair and skin, active ingredients intended to provide a treating action vis-à-vis the skin or hair, mineral fillers or pigments, particles procuring a visual effect or intended for encapsulating active ingredients, exfoliating particles, texture agents, optical brighteners, insect repellents.

Among the opacifying and/or pearlescent agents that can be associated with the composition (C₁) that is the subject matter of this invention, the following can in particular be cited: sodium or magnesium palmitates, stearates or hydroxylstearates, ethylene or polyethylene glycol monostearates or distearates, fatty alcohols, styrene homopolymers and copolymers such as the styrene acrylate copolymer marketed under the name MONTOPOL™ OP1 by the company SEPPIC.

Among the texture agents that can be associated with the composition (C₁) that is the subject matter of this invention, mention may be made of: N-acyl derivatives of amino acids, such as for example the lauroyl lysine marketed under the name AMINOHOPE™LL by the company AJINOMOTO, the octenyl starch succinate marketed under the name DRYFLO™ by the company NATIONAL STARCH, the myristyl polyglucoside marketed by SEPPIC under the name MONTANOV™ 14, cellulose fibres, cotton fibres, chitosan fibres, talc, sericite, mica.

Among the active ingredients that can be associated with the composition (C₁) that is the subject matter of this invention, mention may be made for example of: vitamins and derivatives thereof, in particular the esters thereof, such as retinol (vitamin A) and the esters thereof (retinyl palmitate for example), ascorbic acid (vitamin C) and the esters thereof, ascorbic acid sugar derivatives (such as for example ascorbyl glucoside), tocopherol (vitamin E) and the esters thereof (such as for example tocopherol acetate), vitamin B3 or B10 (niacinamide and derivatives thereof); the compounds showing a skin lightening or depigmenting action, such as for example SEPIWHITE™MSH, arbutin, kojic acid, hydroquinone, VEGEWHITE™, GATULINE™, SYNERLIGHT™, BIOWHITE™, PHYTOLIGHT™, DERMALIGHT™, CLARISKIN™, MELASLOW™, DERMAWHITE™, ETHIOLINE, MELAREST™, GIGAWHITE™, ALBATINE™, LUMISKIN™; the compounds showing a calming action such as SEPICALM™ S, allantoin and bisabolol; anti-inflammatory agents, compounds showing a moisturising action such as for example urea, hydroxyureas, glycerol, polyglycerols, AQUAXYL™, glycerolglucoside; polyphenols extracts such as for example grape extracts, pine extracts, wine extracts, olive extracts; compounds showing a slimming or lipolytic action such as caffeine or derivatives thereof, ADIPOSLIM™, ADIPOLESS™; N-acylated proteins; N-acylated peptides such as for example MATRIXIL™; N-acylated amino acids; partial hydrolysates of N-acylated proteins; amino acids; peptides; total protein hydrolysates, soya bean extracts, for example Raffermine™; wheat extracts, for example TENSINE™ or GLIADINE™; plant extracts, such as plant extracts enriched in tannins, plant extracts enriched in isoflavones or plant extracts enriched in terpenes; fresh or sea water alga extracts; marine extracts in general such as corals; essential waxes; bacterial extracts; ceramides; phospholipids; compounds showing an antimicrobial action or a purifying action, such as for example LIPACIDE™ CBG, LIPACIDE™ UG, SEPICONTROL™ A5; OCTOPIROX™ or SENSIVA™ SC50; the compounds showing an energising or tonic property such as Physiogényl™, panthenol and derivatives thereof such as SEPICAP™ MP; anti-aging agents such as SEPILIFT™ DPHP, LIPACIDE™ PVB, SEPIVINOL™, SEPIVITAL™, MANOLIVA™, PHYTO-AGE™, TIMECODE™; SURVICODE™; anti-photoaging agents; agents protecting the integrity of the dermo-epidermic junction; agents increasing the synthesis of components of the extracellular matrix such as for example collagen, elastins, glycosaminoglycans; agents promoting chemical cell communication such a cytokines or physical cell communication such as integrins; agents creating a “warming” sensation on the skin such as skin microcirculation activators (such as for example nicotinic acid derivatives); or products creating a “cooling” sensation on the skin (such as for example menthol and derivatives); agents improving skin microcirculation, for example veinotonics; draining agents; agents for decongestant purposes such as for example extracts of gingko biloba, ivy, horse chestnut, bamboo, ruscus, butcher's broom, centalla asiatica, fucus, rosemary and willow.

Among the active ingredients that can be associated with the composition (C₁) that is the subject matter of this invention, the following can more particularly be cited: skin tanning or browning agents, such as for example dihydroxyacetone, isatin, alloxan, ninhydrin, glyceraldehyde, mesotartric aldehyde, glutaraldehyde, erythrulose.

Among the non-ionic detergent surfactants that can be associated with the compound (C₁) that is the subject matter of this invention, mention may be made of fatty alcohol ethoxylated derivatives comprising 8 to 12 carbon atoms, fatty acid ethoxylated derivatives comprising 8 to 12 carbon atoms, fatty ester ethoxylated derivatives comprising 8 to 12 carbon atoms, monoglyceride ethyoxylated derivatives comprising 8 to 12 carbon atoms, alkylpolyglucosides of formula (II):

R₂—O—(S)_{y}—H (II)

wherein y represents a decimal number between 1 and 5, S represents the a reducing sugar residue and R₂ represents a saturated or unsaturated, linear or branched alkyl radical, having 5 to 16 carbon atoms, preferably 8 to 14 carbon atoms, or a mixture of compounds of formula (II).

The non-ionic detergent surfactants that can be assoicated with the composition (C₁) that is the subject matter of this invention are more particularly chosen from the elements of the group consisting of caprylyl capryl glucosides, marketed in particular under the brand name ORAMIX™CG 110 by the company SEPPIC, decylglucoside, marketed in particular under the brand name ORAMIX™ NS 10 by the company SEPPIC.

Among the pigments that can be associated with the composition (C₁) that is the subject matter of this invention, mention may be made of titanium dioxide: titanium dioxide, brown iron oxides, yellow iron oxides, black iron oxides or red iron oxides, or white or coloured pearlescent pigments such as Mica-Titanium.

Among the sun filters that can be associated with the composition (C₁) that is the subject matter of this invention, mention may be made of all those featuring in the amended cosmetics directive 76/768/EEC annex VII, such as for example titanium oxide, zinc oxide, cinnamic acid esters such as for example 2-ethylhexyl 4-methoxycinnamate, isopentyl 4-methoxycinnamate, non-ionic benzophenone derivatives, 4-amino benzoic acid esters such as for example 2-ethylhexyl, 4-(dimethylamino)benzoate or amyl 4-(dimethylamino)benzoate.

According to another particular aspect, the subject matter of the invention is a composition (C₁) as defined above, characterised in that the dynamic viscosity thereof measured at a temperature of 20° C., by means of a Brookfield type viscometer is greater than or equal to 30,000 mPa·s and less than or equal to 200,000 mPa·s, more particularly greater than or equal to 40,000 mPa·s and less than or equal to 130,000 mPa·s, and even more particularly greater than or equal to 50,000 mPa·s and less than or equal to 130,000 mPa·s.

When the dynamic viscosity of the composition (C₁) is less than or equal to approximately 100,000 mPa·s at a temperature of 20° C., said dynamic viscosity is measured by means of a Brookfield LVT type viscometer at a speed of 6 revolutions/minute.

When the dynamic viscosity of the composition (C₁) is greater than approximately 100,000 mPa·s at a temperature of 20° C., said dynamic viscosity is measured by means of a Brookfield RVT type viscometer at a speed of 5 revolutions/minute.

The composition (C₁) that is the subject matter of this invention is in particular in the form of a continuous aqueous phase emulsion or microemulsion.

The composition (C₁) that is the subject matter of this invention may also be used for impregnating substrates consisting of synthetic or natural textile fibres, woven or non-woven, or paper, to form articles, such as for example wipes intended for the care, protection or cleansing of the skin, scalp or hair, or such as for example papers for sanitary or household use.

The composition (C₁) that is the subject matter of this invention can be used by application to the skin, hair or scalp, whether it consists of a direct application in the case of a cosmetic, dermocosmetic, dermopharmaceutical or pharmaceutical composition, or an indirect application in the case of a product for the care, protection, cleansing of the body presenting in the form of a textile article, such as for example a wipe, or a paper article such as for example paper for sanitary use, intended to be in contact with the skin, hair or scalp.

The composition (C₁) as defined above is stable over time after a period of storage of at least one month at 20° C. and retains a homogeneous appearance, not showing the appearance of lumps of clusters, after of the same storage period under the same experimental conditions, without it being necessary to incorporate emulsifying surfactants in said composition (C₁).

According to another particular aspect, the subject matter of this invention is a composition (C₁) comprising for 100% of the weight thereof from 0.1% to 10% by weight, more particularly from 0.1% to 5% by weight and even more particularly from 0.5% to 3% by weight, of at least one emulsifying surfactant (EM) chosen from:
fatty acids comprising 14 to 22 carbon atoms,
ethoxylated fatty acids comprising 14 to 22 carbon atoms,
fatty acid esters comprising 14 to 22 carbon atoms and sorbitol,
fatty acid esters comprising 14 to 22 carbon atoms and polyglycerol,
fatty alcohols comprising 14 to 22 ethoxylated carbon atoms,
fatty acid esters comprising 14 to 22 carbon atoms and sucrose,
alkylpolyglycosides of formula (II):

R₃—O—(S)_{z}—H (III)

wherein z represents a decimal number between 1 and 5, S represents a reducing sugar residue and R₃ represents a saturated or unsaturated, linear or branched alkyl radical, having 14 to 22 carbon atoms, preferably 16 to 22 carbon atoms, or a mixture of compounds of formula (III).

In the definition of formula (III) as defined above, z is a decimal number that represents the mean degree of polymerisation of the residue S. When z is an integer number, (S)_{z} is the rank z polymeric residue of the residue S. When z is a decimal number, formula (III) represents a mixture of compounds:

a₁ R₃—O—S—H+a₂ R₃—O—(S)₂—H+a₃ R₃—O—(S)₃—H+ . . . +a_{q} R₃—O—(S)_{q}—H with q representing an integer number between 1 and 10 and in the molar proportions a₁, a₂, a₃, . . . a_{q} such that:

q=1

Σa_{q}=1; a1>0

q=10

In formula (III) as defined above, z is between 1.05 and 5.0 and more particularly between 1.05 and 2.

In formula (III) as defined above, R₃ represents for example the n-tetradecyl radical, the n-hexadecyl radical, the n-octadecyl radical, the n-eicosyl radical or the n-dodecosyl radical.

Reducing sugar means, in the definition of formula (III), the saccharide derivatives that do not have, in the structures thereof, a glucoside bond established set out between an anomeric carbon and the oxygen of an acetal group as defined in the reference publication “Biochemistry”, Daniel Voet/Judith G. Voet, p. 250, John Wyley & Sons, 1990. The oligomeric structure (S)_{z}, may be presented in any form of isomerism, whether it consists of optical isomerism, geometric isomerism or position isomerism; it may also represent a mixture of isomers.

In formula (III) as defined above, the R₃—O— group is bonded to S by the anomeric carbon of the saccharide residue, so as to form an acetal function.

In formula (III) as defined above, S represents more particularly the residue of a reducing sugar chosen from glucose, xylose or arabinose.

According to this other particular aspect, the ratio by weight between the sum of the quantity by weight of anionic polyelectrolyte (PA) and the quantity by weight of xanthan gum (GX) and the quantity by weight of acacia gum (GA), as defined above, and the quantity by weight of the emulsifying agent (EM) is greater than or equal to 1.0, more particularly greater than or equal to 5.0, and even more particularly greater than or equal to 10.0.

According to another aspect, the subject matter of this invention is a method for preparing the composition (C₁) as defined above, comprising:
at least a step a) for preparing a phase (P′₁) by mixing the cross-linked anionic polyelectrolyte (PA), xanthan gum (GX) and acacia gum (GA) in the oil phase (P₁); and
at least a step b) for emulsifying the phase (P′₁) obtained following step a) with the cosmetically acceptable aqueous phase (P₂).

In the method that is the subject matter of the invention, the oil phase (P₁) comprises one or a plurality of oils and/or one or a plurality of waxes as defined above.

In case the oil phase (P₁) does not consist of a single oil or a single wax, the oil phase (P₁) is prepared by mixing the constituent ingredients thereof at a temperature typically between 20° C. and 85° C., and even more particularly at a temperature between 20° C. and 60° C., and by means of any mixing device known to persons skilled in the art, such as for example by means of a mechanical stirring device equipped with an “anchor” type mobile assembly, at stirring speeds of between 50 revolutions/minute and 500 revolutions/minute, more particularly between 50 revolutions/minute and 300 revolutions/minute.

In the method that is the subject matter of the invention as described above, step a) of preparing a phase (P′₁) by mixing cross-linked anionic polyelectrolyte (PA), xanthan gum (GX) and acacia gum (GA) in the oil phase (P₁) can advantageously be implemented at a temperature less than or equal to 85° C. and greater than or equal to 20° C., more particularly at a temperature of less than or equal to 60° C. and greater than or equal to 20° C.

In the method that is the subject matter of the invention as described above, step a) of preparing a phase (P′₁) by mixing cross-linked anionic polyelectrolyte (PA), xanthan gum (GX) and acacia gum (GA) in the oil phase (P₁) can be performed by means of any mixing device known to the person skilled in the art, such as for example by means of a mechanical stirring device equipped with an “anchor” type mobile assembly, at stirring speeds of between 50 revolutions/minute and 500 revolutions/minute, more particularly between 50 revolutions/minute and 300 revolutions/minute, and such as for example by means of a stirring device of the rotor-stator type at stirring speeds of between 100 revolutions/minute and 10,000 revolutions/minute, more particularly between 500 revolutions/minute and 4,000 revolutions/minute.

In the method that is the subject matter of the invention, step b) for emulsifying the phase (P′₁) obtained following step a) with the aqueous phase (P₂) can advantageously be implemented at a temperature of between 20° C. and 90° C., more particularly at a temperature of between 20° C. and 85° C., and even more particularly at a temperature of between 20° C. and 60° C.

In the method that is the subject matter of the invention, step b) for emulsifying the phase (P′₁) obtained following step a) with the aqueous phase (P₂) can be performed by means of any mixing device known to persons skilled in the art, such as for example by means of a mechanical stirring device equipped with an <<anchor>> type mobile assembly, at stirring speeds of between 50 revolutions/minute and 500 revolutions/minute, more particularly between 50 revolutions/minute and 300 revolutions/minute, and such as for example by means of a stirring device of the rotor-stator type at stirring speeds of between 100 revolutions/minute and 10,000 revolutions/minute, more particularly between 500 revolutions/minute and 4,000 revolutions/minute.

In the method that is the subject matter of the invention as described above, the cosmetically acceptable aqueous phase (P₂) comprises water, and optionally one or a plurality of cosmetically acceptable organic solvents as described previously, and from 1% to 25% by weight for 100% of the weight of said cosmetically acceptable aqueous phase (P₂) of at least one salt (S) in presented in a dissolved form and as defined previously.

The cosmetically acceptable aqueous phase (P₂) is prepared by mixing water, and optionally one or a plurality of cosmetically acceptable organic solvents, with at least one salt (S) as described previously, at a temperature of between 20° C. and 85° C., and even more particularly at a temperature of between 20° C. and 60° C., and by means of any mixing device known to persons skilled in the art, such as for example by means of a mechanical stirring device equipped with an <<anchor>> type mobile assembly, at stirring speeds of between 50 revolutions/minute and 500 revolutions/minute, more particularly 20 between 50 revolutions/minute and 300 revolutions/minute.

According to another aspect, the subject matter of this invention is the cosmetic use of the composition (C₁) as defined above for cleansing, protection and/or care of the skin, hair, scalp or mucosa.

Within the scope of this invention, “cosmetic use” means uses of the composition (C₁) intended to improve and/or preserve the external aesthetic appearance of the skin, hair, scalp or mucosa.

According to a more particular aspect, the composition (C₁) that is the subject matter of this invention can be used for cleansing the skin, hair or scalp, and more particularly can be used as a bath or shower gel, as a shampoo. In this particular use, it further comprises at least one non-ionic detergent surfactant as described previously.

According to another more particular aspect, the composition (C₁) that is the subject matter of this invention can be used for caring for or for protecting the skin, such as for example as a cream, milk or lotion for caring for or protecting the face, hands and body. According to this particular aspect, the composition (C₁) can also be used more particularly as a product for protecting the skin against the rays of the sun, as a skin makeup product, as a product protecting the skin against skin aging, as a skin moisturising product, as a product for the cosmetic treatment of acne and/or blackheads and/or comedones.

The following examples illustrate the invention without however limiting it.

### 1.1 Preparation of aterpolymer of ammonium 2-methyl 2-[(1-oxo 2-propenyl)amino] 1-propanesulfonate, N,N-dimethyl acrylamide and tetraethoxylated lauryl methacrylate [AMPS/DMAM/MAL(4OE) 77.4/19.2/3.4 molar], Cross-Linked with trimethylol propanetriacrylate (TMPTA) [Example According to the Invention].

592 g of an aqueous solution containing 15% by weight of ammonium 2-methyl 2-[(1-oxo 2-propenyl)amino] 1-propanesulfonate in a tert-butanol/water mixture (97.5/2.5 by volume), 10.1 g of N,N dimethyl acrylamide, 4.2 g of tetraethoxylated lauryl methacrylate and 0.75 g of trimethylol propanetriacrylate are loaded into a reactor maintained at 25° C. under stirring.

After sufficient time to achieve satisfactory homogenisation of the solution, the solution is deoxygenated by bubbling nitrogen heated to 70° C. 0.42 g of dilauroyl peroxide is then added and the reaction medium is then maintained at 70° C. for approximately 60 minutes at 70° C. and 2 hours at 80° C.

After cooling, the powder that formed during polymerisation is filtered and dried to obtain the required product, hereinafter referred to as “Polyelectrolyte 1”.

### 1.2. Preparation of a terpolymer of ammonium 2-methyl 2-[(1-oxo 2-propenyl)amino] 1-propanesulfonate, 2-hydroxyethylacrylate and tetraethoxylated lauryl methacrylate [AMPS/HEA/MAL(4OE) 77.4/19.2/3.4 molar], Cross-Linked with trimethylol propanetriacrylate (TMPTA) [Comparative Example].

Using the operating conditions of the method described in example 1.1 above, the quantity required of an aqueous solution containing 15% by weight of ammonium 2-methyl 2-[(1-oxo 2-propenyl)amino] 1-propanesulfonate in a tert-butanol/water mixture (97.5/2.5 by volume) so as to introduce 77.4 molar equivalents of ammonium 2-methyl 2-[(1-oxo 2-propenyl)amino] 1-propanesulfonate, the quantity required by weight of 2-hydroxyethylacrylate so as to introduce 19.2 molar equivalents of 2-hydroxyethylacrylate, the quantity required by weight of tetraethoxylated lauryl methacrylate so as to introduce 3.4 molar equivalents of tetraethoxylated lauryl methacrylate, and the quantity required by weight of trimethylol propanetriacrylate so as to obtain the same molar proportion of trimethylol propanetriacrylate as in example 1.1 are loaded into a reactor maintained at 25° C. under stirring.

After sufficient time to achieve satisfactory homogenisation of the solution, the solution is deoxygenated by bubbling nitrogen heated to 70° C. 0.42 g of dilauroyl peroxide is then added and the reaction medium is then maintained for approximately 60 minutes at 70° C. and 2 hours at 80° C.

After cooling, the powder that formed during polymerisation is filtered and dried to obtain the required product, hereinafter referred to as “Polyelectrolyte 2”.

### 1.3. Preparation of a copolymer of ammonium 2-methyl 2-[(1-oxo 2-propenyl) aminol 1-propanesulfonate and tetraethoxylated lauryl methacrylate [AMPS/MAL(4OE) 95/5 molar], Cross-Linked with trimethylol propanetriacrylate (TMPTA) [Comparative Example].

Using the operating conditions of the method described in the example 1.1 above, the quantity required by weight of an aqueous solution containing 15% by weight of ammonium 2-methyl 2-[(1-oxo 2-propenyl)amino] 1-propanesulfonate in a tert-butanol/water mixture (97.5/2.5 by volume) so as to introduce 95 molar equivalents of ammonium 2-methyl 2-[(1-oxo 2-propenyl)amino] 1-propanesulfonate, the quantity required by weight of tetraethoxylated lauryl methacrylate so as to introduce 5 molar equivalents of tetraethoxylated lauryl methacrylate, and the quantity required by weight of trimethylol propanetriacrylate so as to obtain the same molar proportion of trimethylol propanetriacrylate as in example 1.1 are loaded into a reactor maintained at 25° C. under stirring.

After sufficient time to achieve satisfactory homogenisation of the solution, the solution is deoxygenated by bubbling nitrogen heated to 70° C. 0.42 g of dilauroyl peroxide is then added and the reaction medium is then maintained for approximately 60 minutes at 70° C. and 2 hours at 80° C.

After cooling, the powder that formed during polymerisation is filtered and dried to obtain the required product, hereinafter referred to as “Polyelectrolyte 3”.

### 1.4. Preparation of an ammonium 2-methyl 2-[(1-oxo 2-propenyl)aminol 1-propanesulfonate and 2-hydroxyethylacrylate copolymer [AMPS/HEA 90/10 molar], Cross-Linked with trimethylol propanetriacrylate (TMPTA) [Comparative Example].

Using the operating conditions of the method described in the above example 1.1, the quantity required by weight of an aqueous solution containing 15% by weight of ammonium 2-methyl 2-[(1-oxo 2-propenyl)amino] 1-propanesulfonate in a tert-butanol/water mixture (97.5/2.5 by volume) so as to introduce 90 molar equivalents of ammonium 2-methyl 2-[(1-oxo 2-propenyl)amino] 1-propanesulfonate, the quantity required by weight of 2-hydroxyethylacrylate so as to introduce 10 molar equivalents of 2-hydroxyethylacrylate, and the necessary quantity by weight of trimethylol propanetriacrylate so as to obtain the same molar proportion of trimethylol propanetriacrylate as in example 1.1 are loaded into a reactor maintained at 25° C. under stirring.

After sufficient time to achieve satisfactory homogenisation of the solution, the solution is deoxygenated by bubbling nitrogen heated to 70° C. 0.42 g of dilauroyl peroxide is then added and the reaction medium is then maintained for approximately 60 minutes at 70° C. and 2 hours at 80° C.

After cooling, the powder that formed during polymerisation is filtered and dried to obtain the required product, hereinafter referred to as “Polyelectrolyte 4”.

### 2-1 Preparation of Oil-in-Water Emulsions

Six oil-in-water emulsions according to the invention, referenced (E₁) to (E₆), of which the proportions by weight of the constituents thereof are recorded in table 1, and nineteen oil-in-water emulsions referenced (F₀) to (F₁₅) and (G₁) to (G₃) according to the prior art, of which the proportions by weight of the constituents thereof are recorded in table 2 below, are prepared using for each the following method:
the oil phase is poured into a beaker at a temperature of 20° C., then the polyelectrolyte tested, the xanthan gum and/or the acacia gum are dispersed progressively and according to the circumstances successively under mechanical stirring at 80 revolutions/minute;
the aqueous phase comprising the water and if necessary the quantity by weight of salt is poured into a beaker at a temperature of 20° C.;
the content of the beaker comprising the oil phase, comprising the polyelectrolyte tested, the xanthan gum and acacia gum is progressively added to the aqueous phase at a temperature of 20° C., under mechanical stirring by means of a defloculator, at 1200 revolutions per minute.
the mixture thus obtained is maintained under stirring for a period of 10 minutes, then drained to obtain the oil-in-water emulsions (E₁) to (E₆) according to the invention and the oil-in-water emulsions (F₀) to (F₁₅) and (G₁) to (G₃) according to the prior art.

TABLE 1

| | Emulsion | | | | | |
|---|---|---|---|---|---|---|
| | (E₁) | (E₂) | (E₃) | (E₄) | (E₅) | (E₆) |
| Oil phase: | | | | | | |
| C8-C10 triglycerides | 15% | 15% | 15% | 15% | 15% | 15% |
| Stabilising system: | | | | | | |
| Polyelectrolyte 1 | 2.0% | 2.0% | 2.0% | 2.0% | 2.0% | 2.0% |
| Keltrol ™ CG-T⁽²⁾ | 0.225% | 0.225% | 0.375% | 0.375% | 0.125% | 0.125% |
| Efficacia ™ M⁽³⁾ | 0.275% | 0.275% | 0.125% | 0.125% | 0.375% | 0.375% |
| Aqueous phase: | | | | | | |
| Water | Qs. 100% | Qs. 100% | Qs. 100% | Qs. 100% | Qs. 100% | Qs. 100% |
| Sodium chloride | 2% | 4% | 2% | 4% | 2% | 4% |
| Euxyl PE910⁽¹⁾ | 1% | 1% | 1% | 1% | 1% | 1% |

TABLE 2

| | Emulsion | | | | | |
|---|---|---|---|---|---|---|
| | (F₀) | (F₁) | (F₂) | (F₃) | (F₄) | (F₅) |
| Oil phase: | | | | | | |
| C8-C10 triglycerides | 15% | 15% | 15% | 15% | 15% | 15% |
| Stabilising system: | | | | | | |
| Polyelectrolyte 1 | 2.5% | 2.5% | 2.5% | 2.0% | 2.0% | 2.0% |
| Keltrol ™ CG-T⁽²⁾ | 0% | 0% | 0% | 0.5% | 0.5% | 0.5% |
| Efficacia ™ M⁽³⁾ | 0% | 0% | 0% | 0% | 0% | 0% |
| Aqueous phase: | | | | | | |
| Water | Qs. 100% | Qs. 100% | Qs. 100% | Qs. 100% | Qs. 100% | Qs. 100% |
| Sodium chloride | 0% | 2% | 4% | 0% | 2% | 4% |
| Euxyl PE910⁽¹⁾ | 1% | 1% | 1% | 1% | 1% | 1% |
| | Emulsion | | | | | |
| | (F6) | (F7) | (F8) | (G1) | (G2) | (G3) |
| Oil phase: | | | | | | |
| C8-C10 triglycerides | 15% | 15% | 15% | 15% | 15% | 15% |
| Stabilising system: | | | | | | |
| Polyelectrolyte 1 | 20% | 2.0% | 2.0% | 2.0% | 2.0% | 2.0% |
| Keltrol ™ CG-T⁽²⁾ | 0% | 0% | 0% | 0.225% | 0.375% | 0.125% |
| Efficacia ™ M⁽³⁾ | 0.5% | 0.5% | 0.5% | 0.275% | 0.125% | 0.375% |
| Aqueous phase: | | | | | | |
| Water | Qs. 100% | Qs. 100% | Qs. 100% | Qs. 100% | Qs. 100% | Qs. 100% |
| Sodium chloride | 0% | 2% | 4% | 0% | 0% | 0% |
| Euxyl PE910⁽¹⁾ | 1% | 1% | 1% | 1% | 1% | 1% |

Emulsion

| | (F₉) | (F₁₀) | (F₁₁) | (F₁₂) |
|---|---|---|---|---|
| Oil phase: | | | | |
| C8-C10 triglycerides | 15% | 15% | 15% | 15% |
| Stabilising system: | | | | |
| Polyelectrolyte 2 | 2% | 2% | 0% | 0% |
| Polyelectrolyte 3 | 0% | 0% | 2% | 2% |
| Keltrol ™ CG-T⁽²⁾ | 0.225% | 0.225% | 0.225% | 0.225% |
| Efficacia ™ M⁽³⁾ | 0.275% | 0.275% | 0.275% | 0.275% |
| Aqueous phase: | | | | |
| Water | Qs. 100% | Qs. 100% | Qs. 100% | Qs. 100% |
| Sodium chloride | 2% | 4% | 2% | 4% |
| Euxyl PE910⁽¹⁾ | 1% | 1% | 1% | 1% |

| | Emulsion | | |
|---|---|---|---|
| | (F₁₃) | (F₁₄) | (F₁₅) |
| Oil phase: | | | |
| C8-C10 triglycerides | 15% | 15% | 15% |
| Stabilising system | | | |
| Polyelectrolyte 1 | 0% | 0% | 2% |
| Polyelectrolyte 4 | 2% | 2% | 0% |
| Keltrol ™ CG-T⁽²⁾ | 0.225% | 0.225% | 0.225% |
| Efficacia ™ M⁽³⁾ | 0.275% | 0.275% | 0.275% |
| Aqueous phase: | | | |
| Water | Qs. 100% | Qs. 100% | Qs. 100% |
| Sodium chloride | 2% | 4% | 10% |
| Euxyl PE910⁽¹⁾ | 1% | 1% | 1% |

⁽¹⁾Euxyl ™ PE910 is a mixture of Phenoxyethanol and Ethylhexylglycerine marketed by the company Schülke & Mayr
⁽²⁾Keltrol ™ CG-T is the xanthan gum marketed by the company CP Kelco
⁽³⁾Efficacia ™ M is the acacia gum marketed by the company CNI

### 2-2 Demonstration of the Properties and Characteristics of the Oil-in-Water Emulsions According to the Invention Compared to Oil-in-Water Emulsions

The formulations (E₁) to (E₆) and the formulations (F₀) to (F₁₅) and (G₁) to (G₃) previously prepared are then assessed as follows:
Measurement of the dynamic viscosity (μ in mPa·s) at 20° C. after 7 days and then one year of storage at 20° C. by means of a Brookfield LVT type viscometer at a speed of 6 revolutions/minute (V6), when said dynamic viscosity is less than or equal to approximately 100,000 mPa·s, equipped with a suitable mobile assembly, or by means of a Brookfield RVT type viscometer at a speed of 5 revolutions/minute (V5), when said dynamic viscosity is greater than 100,000 mPa·s, equipped with a suitable mobile assembly.
Visual assessment of the appearance after a storage period of three months at 20° C.

The oil-in-water emulsions (E₁) to (E₆) according to the invention and the comparative oil-in-water emulsions (F₀) to (F₁₅) and (G₁) to (G₃) thus prepared are then stored in an insulated climatic chamber regulated at a temperature of 20° C. for 7 days. After this period of 7 days and for each oil-in-water emulsion:
the visual appearance is observed,
the dynamic viscosity is measured at 20° C.,
the oil-in-water emulsions are then replaced and stored in the same insulated climatic chamber regulated at a temperature of 20° C. for up to 3 months. After a period of 3 months, each emulsion is removed from the climatic chamber to observe the appearance thereof,
the oil-in-water emulsions are then replaced and stored in the same insulated climatic chamber regulated at a temperature of 20° C. so that the total storage period at 20° C. is one year as from the date of preparation thereof. At the end of this total period of one year, each emulsion is removed from the climatic chamber to measure the dynamic viscosity thereof.

The results obtained are recorded in table 4 below [(+): homogeneous appearance, (++): homogeneous and smooth appearance, (−): presence of lumps and clusters, (nd): not determined].

TABLE 4

| | Emulsion | | | | |
|---|---|---|---|---|---|
| | (G₁) | (E₁) | (E₂) | (G₂) | (E₃) |
| Visual appearance after 7 days at 20° C. | ++ | ++ | ++ | ++ | ++ |
| μ after 7 days at 20° C. | 73,000 | 87,500 | 66,000 | 87,500 | 100,000 |
| μ after 1 year at 20° C. | 50,700 | 52,500 | 49,000 | 55,000 | 69,500 |
| Visual appearance after 3 months at | ++ | ++ | ++ | ++ | ++ |
| 20° C. | | | | | |
| | Emulsion | | | | |
| | (E₄) | (G₃) | (E₅) | (E₆) | (F₀) |
| Visual appearance after 7 days at 20° C. | ++ | ++ | ++ | ++ | + |
| μ after 7 days at 20° C. | 78,500 | 62,000 | 79,500 | 53,000 | 110,000* |
| μ after 1 year at 20° C. | 66,000 | 55,000 | 52,500 | 42,500 | nd |
| Visual appearance after 3 months at | ++ | ++ | ++ | ++ | + |
| 20° C. | | | | | |
| | Emulsion | | | | |
| | (F₁) | (F₂) | (F₃) | (F₄) | (F₅) |
| Visual appearance after 7 days at 20° C. | — | — | — | — | — |
| μ after 7 days at 20° C. | 100,000 | 95,000 | 90,000 | 98,000 | 77,000 |
| μ after 1 year at 20° C. | 89,500 | 111,000* | 82,000 | 76,500 | 56,500 |
| Visual appearance after 3 months at 20° C. | — | — | — | — | — |
| | Emulsion | | | | |
| | (F₆) | (F₇) | (F₈) | (F₉) | (F₁₀) |
| Visual appearance after 7 days at 20° C. | — | — | — | — | — |
| μ after 7 days at 20° C. | 100,000 | 61,000 | 53,000 | 58,000 | 53,000 |
| μ after 1 year at 20° C. | 55,000 | 53,000 | 44,000 | — | — |
| Visual appearance after 3 months at 20° C. | — | — | — | | |
| | Emulsion | | | | |
| | (F₁₁) | (F₁₂) | (F₁₃) | (F₁₄) | (F₁₅) |
| Visual appearance after 7 days at 20° C. | — | — | — | — | — |
| μ after 7 days at 0° C. | 30,000 | 35,000 | 6,300 | 4,580 | 43,000 |
| Visual appearance after 3 months at 20° C. | — | — | — | — | — |

*dynamic viscosity measured at 20° C. with the Brookfield RVT viscometer, V5.

### 2-3 Analysis of the Results

The results are deemed to be satisfactory when the visual appearance of an oil-in-water emulsion is deemed to be homogeneous and smooth after a storage period of three months at 20° C. of said oil-in-water emulsion, and when the dynamic viscosity thereof measured at 20° C., by means of a Brookfield LVT type viscometer at a speed of 6 revolutions/minute, equipped with the suitable mobile assembly, is greater than or equal to 30,000 mPa·s.

The emulsions (E₁) to (E₆) according to the invention have a smooth appearance, devoid of lumps and clusters, even following a prolonged storage period of 3 months at 20° C.

The results obtained for the comparative emulsions (F₁) and (F₂) show that, when the stabilising system of the oil-in-water emulsion consists solely of polyelectrolyte 1 in the presence of a quantity of 2% and 4% of sodium chloride, oil-in-water emulsions having a homogeneous and smooth appearance after a storage period of 7 days at 20° C. are not obtained. Moreover, comparison of the emulsion (F₀) with the emulsions (F₁) and (F₂) shows that the presence of sodium chloride causes a degradation of the appearance of said emulsion.

Comparison of the behaviour in storage of the emulsion (G₁) with that of emulsions (E₁) and (E₂) according to the invention, (G₁), (E₁) and (E₂) comprising the same stabilising system and (G₁) differing through the absence of sodium chloride, shows that the presence of sodium chloride does not cause degradation of the appearance of said emulsion.

The results obtained for the comparative emulsions (F₃), (F₄) and (F₅) show that, when the stabilising system of the oil-in-water emulsion consists of the combination of polyelectrolyte 1 in a quantity by weight of 2% and only xanthan gum, in the presence respectively of 0%, 2% and 4% of NaCl, oil-in-water emulsions (F₃), (F₄) and (F₅) having a homogeneous and smooth appearance after a storage period of 7 days at 20° C. are not obtained.

The results obtained for the comparative emulsions (F₆), (F₇) and (F₈) show that, when the stabilising system of the oil-in-water emulsion consists of the combination of polyelectrolyte 1 in a quantity by weight of 2% and only acacia gum, in the presence respectively of 0%, 2% and 4% of NaCl, oil-in-water emulsions (F₆), (F₇) and (F₈) having a homogeneous and smooth appearance after a storage period of 7 days at 20° C. are not obtained.

The comparative emulsions (F₉) and (F₁₀) comprising the polyelectrolyte 2, and respective sodium chloride quantities of 2% and 4%, show the presence of lumps after a storage period 7 days at 20° C.

The comparative emulsions (F₁₁) and (F₁₂) comprising the polyelectrolyte 3, and respective sodium chloride quantities of 2% and 4%, show the presence of lumps after a storage period of 7 days at 20° C.

The comparative emulsions (F₁₃) and (F₁₄) comprising the polyelectrolyte 4, and respective sodium chloride quantities of 2% and 4%, show the presence of lumps after a storage period of 7 days at 20° C. The emulsions (F₁₃) and (F₁₄) do not make it possible to obtain the required viscosity level, namely a minimum dynamic viscosity of 30,000 mPa·s (measured at 20° C. by means of a Brookfield LVT type viscometer at a speed of 6 revolutions/minute).

The comparative emulsion (F₁₅) comprising the polyelectrolyte 1, xanthan gum and acacia gum, in a quantity by weight of sodium chloride of 10% (that is to say 12.1% by weight of sodium chloride in the aqueous phase alone) shows the presence of lumps after a storage period of 7 days at 20° C.

A comparison of the results obtained for the oil-in-water emulsions (E₁) to (E₆) according to the invention and for the comparative oil-in-water emulsions (F₁) to (F₁₅) and (G₁) to (G₃) clearly demonstrates that the improvement in the appearance of the oil-in-water emulsions, rich in salt, and keeping a high level of viscosity, could not be deduced from the results associated with the comparative oil-in-water emulsions.

### 3 Preparation of Oil-in-Water Emulsions of the Restructuring “Rinse Off” Cream Mask Type for Stressed and Weakened Hair According to the Invention and According to the Prior Art

An oil-in-water emulsion according the prior art referenced (F₁₆) and an oil-in-water emulsion according to the invention referenced (E₇) are prepared, the proportions by weight of the constituents thereof are recorded in table 5 below.

The common preparation method for the oil-in-water emulsions (F₁₆) and (E₇) is as follows:
the oil phase is prepared by pouring into a beaker, at a temperature of 80° C. progressively and successively, Lanol™P, Lanol™99, jojoba oil and Montanov™82, and then the polyelectrolyte (PA₁) and xanthan gum, and acacia gum where applicable, are dispersed progressively, under mechanical stirring at 80 revolutions per minute;
the aqueous phase comprising the water onto which the butylene glycol, N-cocoyl amino acids, PECOSIL™SPP 50, AMONYL™DM, SEPICIDE™HB and SEPICIDE™CI are progressively and successively poured, is prepared in a beaker at a temperature of 20° C.;
the content of the beaker comprising the oil phase, the polyelectrolyte (PA₁) and xanthan gum, and acacia gum where applicable, is progressively added to the aqueous phase at a temperature of 80° C., under mechanical stirring by means of a deflocculator, at 1200 revolutions per minute;
the mixture thus obtained is maintained under stirring for a period of 10 minutes, and then drained to obtain the oil-in-water emulsions (F₁₆) and (E₇), the compositions by weight of which are referenced in the following table 5:

TABLE 5

| Emulsion | (F₁₆) | (E₇) |
|---|---|---|
| Oil phase: | | |
| Jojoba oil | 1% | 1% |
| Lanol ™P⁽⁴⁾ | 6% | 6% |
| Lanol ™99⁽⁵⁾ | 5% | 5% |
| Montanov ™82⁽⁶⁾ | 3% | 3% |
| Stabilising system: | | |
| Polyelectrolyte (PA1) | 1% | 1% |
| Keltrol ™CG-T⁽²⁾ | 1% | 0.5% |
| Efficacia ™M⁽³⁾ | 0% | 0.5% |
| Aqueous phase: | | |
| water | Qs. 100% | Qs. 100% |
| butylene glycol | 3% | 3% |
| N-cocoyl amino acids | 0.7% | 0.7% |
| PECOSIL ™SPP 50⁽⁷⁾ | 0.75% | 0.75% |
| AMONYL ™DM⁽⁸⁾ | 1% | 1% |
| SEPICIDE ™HB⁽⁹⁾ | 0.3% | 0.3% |
| SEPICIDE ™CI⁽¹⁰⁾ | 0.2% | 0.2% |

(4): LANOL™ P is a glycol palmitate used as an additive with stabilising effect, and marketed by the company SEPPIC.
(5): LANOL™ 99 is isononyl isononanoate marketed by the company SEPPIC.
(6): MONTANOV™ 82 is an emulsifying agent based on cetearyl alcohol and cocoylglucoside.
(7): PECOSIL™SPP 50 is a potassium dimethicone PEG-7 panthenyl phosphate, marketed by the company PHOENIX.
(8): AMONYL DM is a cationic surfactant presented in the form of a quaternary ammonium salt, the INCI name of which is “Polyquaternium 82”, and marketed by the company SEPPIC.
(9): SEPICIDE™ HB, which is a mixture of phenoxyethanol, methylparaben, ethylparaben, propylparaben and butylparaben, is a preservative marketed by the company SEPPIC.
(10): SEPICIDE™ CI, urea imadazolidine, is a preservative marketed by the company SEPPIC.

The oil-in-water emulsion (F₁₆) according to the prior art and the emulsion (E₇) according to the invention are evaluated according to the experimental protocol described in paragraph 2-2 above of the present patent application.

The results obtained for the oil-in-water emulsion (F₁₆) according to the prior art and the results obtained for the emulsion (E₇) according to the invention are recorded in table 6 below.

TABLE 6

| Emulsion | (F₁₆) | (E₇) |
|---|---|---|
| Visual appearance after 7 days at 20° C. | − | ++ |
| μ after 7 days at 20° C. (Brookfield LVT, V6) in | 100,000 | 71,000 |
| mPa · s | | |
| Visual appearance after 3 months at 20° C. | − | ++ |

The oil-in water emulsion (E₇) according to the invention has a smooth appearance, free from lumps and clusters, after a prolonged storage period of 3 months at 20° C., whereas the oil-in-water emulsion (F₁₆) according to the prior art has a heterogeneous appearance with the presence of lumps and clusters after the same storage period under the same operating conditions.

### 4.1: Face Mask Gel-Cream

### Formula

| | | |
|---|---|---|
| A | Simmondsia chinensis seed oil | 14.1% |
| | C12-C15 alkyl benzoate | 6.7% |
| | Cyclopentasiloxane | 4.2% |
| | DL alpha tocopherol | 0.10% |
| B | Maris Aqua | 70.85% |
| C | Polyelectrolyte 1 | 2% |
| | Keltrol ™CG-T | 0.45% |
| | Efficacia ™M | 0.55% |
| D | Euxyl PE9010 | 1% |
| | Fragrance | 0.1% |

### Operating Method

Mix the constituents of the oil phase A at a temperature of 80° C. under stirring.

Next successively add the ingredients of phase C successively at 80° C. and under stirring.

Prepare the aqueous phase B and heat it to 80° C. under stirring.

Add the aqueous phase B progressively to the mixture of phases A+C then emulsify by means of a stirrer equipped with a Silverson rotor-stator mobile assembly.

Then cool to 25° C. then add phase D.

Adjust the pH to 6.

Appearance after 1 day at 20° C.: homogeneous compact cream.
Dynamic viscosity after 1 day at 20° C.: 124,000 mPa·s (Brookfield RVT, M7, V5).
Appearance after 7 days at 20° C.: homogeneous compact cream.
Dynamic viscosity after 7 days at 20° C.: 112,000 mPa·s (Brookfield RVT, M7, V5).
Appearance after 1 month at 20° C.: homogeneous compact cream.
Dynamic viscosity after 1 month at 20° C.: 112,000 mPa·s (Brookfield RVT, M7, V5).

### 4.2: Face Mask Gel-Cream

### Formula

| | | |
|---|---|---|
| A | Triglycerides 4555 (C8C10) | 9% |
| | C12-C15 alkyl benzoate | 4% |
| | Isohexadecane | 2% |
| | DL alpha-tocopherol | 0.10% |
| B | Maris aqua | qsp 100% |
| C | Polyelectrolyte 1 | 1.3% |
| | Keltrol ™CG-T | 0.315% |
| | Efficacia ™M | 0.385% |
| D | Euxyl PE9010 | 1% |
| | Fragrance | 0.1% |

### Operating Method

Mix the constituents of the oil phase A at a temperature of 80° C. under stirring.

Then successively add the ingredients of phase C at 80° C. and under stirring.

Prepare the aqueous phase B and heat it to 80° C. under stirring.

Add the aqueous phase B progressively to the mixture of phases A+C then emulsify by means of a stirrer equipped with a Silverson rotor-stator mobile assembly.

Then cool to 25° C. then add phase D.

Appearance after 1 day at 20° C.: homogeneous compact cream.
Dynamic viscosity after 1 day at 20° C.: 71,000 mPa·s (Brookfield LVT, M4, V6).
Appearance after 7 days at 20° C.: homogeneous compact cream.
Dynamic viscosity after 7 days at 20° C.: 75,000 mPa·s (Brookfield LVT, M4, V6).
Appearance after 1 month at 20° C.: homogeneous compact cream.
Dynamic viscosity after 1 month at 20° C.: 73,000 mPa·s (Brookfield LVT, M4, V6).
Appearance after 3 months at 20° C.: homogeneous compact cream.
Dynamic viscosity after 3 months at 20° C.: 70,400 mPa·s (Brookfield LVT, M4, V6).

### 4.3: Organomineral Sun Spray

### Formula

| | | |
|---|---|---|
| A | Isodecyl neopentanoate | 20% |
| | Cyclodimethicone | 5% |
| | Ethylhexylmethoxycinnamate | 6% |
| | Butyl methoxydibenzoylmethane | 3% |
| | DL alpha tocopherol | 0.05% |
| B | Water | qsp 100% |
| | Tetrasodium EDTA | 0.2% |
| | Glycerin | 7% |
| | Phenyl benzimidazole sulfonic acid | 3% |
| | (salified with the necessary molar quantity of soda) | |
| C | Polyelectrolyte 1 | 1.3% |
| | Keltrol ™CG-T | 0.315% |
| | Efficacia ™M | 0.385% |
| D | SEPICIDE ™HB | 1% |
| | Fragrance | 0.1% |

### 4.4: Body Cream

### Formula

| | |
|---|---|
| Triglycerides 4555 (C8C10) | 12% |
| C12-C15 alkyl benzoate | 5.3% |
| Isohexadecane | 2.7% |
| Cetyl alcohol | 2% |
| DL alpha tocopherol | 0.10% |
| Polyelectrolyte 1 | 1.5% |
| Keltrol ™CG-T | 0.25% |
| Efficacia ™M | 0.25% |
| Water | qsp 100% |
| Givobio ™GZn | 1% |
| Sepicalm ™S | 3% |
| Euxyl PE9010 | 1% |
| Fragrance | 0.1% |

Euxyl PE9010 (INCI name: Phenoxyethanol & Ethylhexyl Glycerin): composition used as a preservative
GIVOBIO™ GZn (INCI name: Zinc Gluconate) is a composition marketed by the company SEPPIC.
Maris Aqua: seawater with 8% sodium chloride.
SEPICALM™ S: (INCI name: Sodium Cocoyl Aminoacids And Sarcosine And Potassium Aspartate And Magnesium Aspartate) is an anti-inflammatory composition marketed by the company SEPPIC.
SEPICIDE™ HB (INCI name: Phenoxyethanol/Methylparaben/Ethylparaben/Propylparaben/Butylparaben) is a preservative containing phenoxyethanol, marketed by the company SEPPIC.

1. Composition (C₁) presented in the form of an emulsion of the oil-in-water type emulsion, which comprises for 100% of the weight thereof:
from 5% to 55% by weight, more particularly from 7% to 30% by weight and even more particularly from 10% to 20% by weight, of an oil phase (P₁) consisting of at least one oil and optionally at least one wax;
from 0.06% to 4.5% by weight of at least one cross-linked anionic polyelectrolyte (PA) resulting from the polymerisation of partially or completely salified 2-methyl 2-[(1-oxo 2-propenyl)amino] 1-propane sulfonic acid, with at least one neutral monomer chosen from the N,N-dialkyl acrylamides, wherein each of the alkyl groups comprises between one and four carbon atoms, and at least one monomer of formula (I): 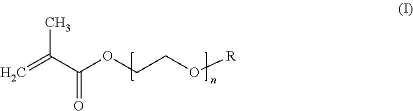
wherein R represents a linear or branched alkyl radical comprising from eight to twenty carbon atoms and n represents a number greater than or equal to one and less than or equal to twenty, in the presence of at least one cross-linking agent;
from 0.0025% to 1% by weight of a xanthan gum (GX);
from 0.0025% to 1% by weight of an acacia gum (GA);
from 38.5% to 94.935% by weight of a cosmetically acceptable aqueous phase (P₂), said aqueous phase (P₂) comprising, for 100% of the weight thereof, from 1% to 10% by weight of at least one salt (S) presented in a dissolved form;
said composition (C₁) furthermore being characterised in that the ratio by weight between the xanthan gum (GX) and the acacia gum (GA) is greater than or equal to 1/3 and less than or equal to 3/1.

2. Composition (C₁) as defined in claim 1, characterised in that said cross-linked anionic polyelectrolyte (PA) comprises for 100% molar of the constituent monomers thereof:
from 20% molar to 80% molar of monomeric units from partially or completely salified 2-methyl 2-[(1-oxo 2-propenyl)amino] 1-propanesulfonic acid;
from 15% molar to 75% molar of monomeric units issuing from a neutral monomer chosen from the N,N-dialkyl acrylamides, wherein each of the alkyl groups comprises between 1 and 4 carbon atoms;
from 0.5% to 5% molar of monomeric units from a monomer of formula (I) as defined above.

3. Composition (C₁) as defined in claim 1, characterised in that in said cross-linked anionic polyelectrolyte (PA), said neutral monomer is N,N-dimethyl acrylamide.

4. Composition (C₁) as defined in claim 1, characterised in that, in said cross-linked anionic polyelectrolyte (PA) said monomer of formula (I) is tetraethoxylated lauryl methacrylate.

5. Composition (C₁) as defined in claim 1, characterised in that said cross-linked anionic polyelectrolyte (PA) is a terpolymer of partially salified 2-methyl 2-[(1-oxo 2-propenyl)amino] 1-propane sulfonic acid in the form of ammonium salt, N,N-dimethyl acrylamide and tetraethoxylated lauryl methacrylate, cross-linked with trimethylol propanetriacrylate.

6. Composition (C₁) as defined in claim 1, characterised in that said cross-linked anionic polyelectrolyte (PA) comprises, for 100% molar:
from 60% molar to 80% molar of monomeric units from 2-methyl 2-[(1-oxo 2-propenyl)amino] 1-propane sulfonic acid partially salified in ammonium form,
from 15% molar to 39.5% molar of monomeric units from N,N-dimethyl acrylamide, and
from 0.5% molar to 5% molar of monomeric units from tetraethoxylated lauryl methacrylate.

7. Composition (C₁) as defined in claim 1, characterised in that the salt (S) is an inorganic salt consisting of a cation that is the ammonium ion or a metal cation and of an anion selected from the elements of the group consisting of the halides, carbonates, bicarbonates, phosphates, nitrates, borates and sulfates.

8. Composition (C₁) as defined in claim 1, characterised in that the salt (S) is an organic salt consisting of a cation that is the ammonium ion or a metal cation and of an organic anion that is an organic compound having at least one carboxylic acid function in carboxylate form or at least one sulfonic acid function in sulfonate form or at least one sulfate function.

9. Composition (C₁) as defined in claim 8, characterised in that the salt (S) is an organic salt selected from the elements of the group consisting of sodium glycolate, sodium citrate, sodium salicylate, sodium lactate, sodium gluconate, zinc gluconate, manganese gluconate, copper gluconate and magnesium aspartate.

10. Composition (C₁) as defined in claim 8, characterised in that the salt (S) is an organic salt selected from sodium 2-phenyl benzimidazole-5 sulfonate or sodium 4-hydroxy 2-methoxy 5-(oxo-phenylmethyl)benzene sulfonate.

11. Composition (C₁) as defined in claim 1, characterised in that the dynamic viscosity thereof measured at a temperature of 20° C., by means of a Brookfield type viscometer is greater than or equal to 30,000 mPa·s and less than or equal to 200,000 mPa·s.

12. Method for preparing a composition (C₁) as defined in claim 1, characterised in that it comprises:
at least a step a) for preparing a phase (P′₁) by mixing the cross-linked anionic polyelectrolyte (PA), xanthan gum (GX) and acacia gum (GA) in the oil phase (P₁); and
at least a step b) for emulsifying the phase (P₁) obtained following step a), with the cosmetically acceptable aqueous phase (P₂).

13. Cosmetic use of the composition (C₁) as defined in claim 1 for the cleansing, protection and/or care of the skin, hair, scalp or mucosa.
